(19) United States
(12) Patent Application Publication
(10) Pub. No.: US 2023/0277077 A1
(43) Pub. Date: Sep. 7, 2023

(54) METHOD, APPARATUS AND SYSTEM FOR ROBUST SUBCUTANEOUS VASCULAR EVALUATION

(71) Applicant: Toronto Metropolitan University, Toronto (CA)

(72) Inventors: Alexandre DOUPLIK, North York (CA); Guennadi SAIKO, Mississauga (CA)

(21) Appl. No.: 18/180,118

(22) Filed: Mar. 7, 2023

(30) Foreign Application Priority Data
Mar. 7, 2022 (CA) 3151180

(51) Int. Cl. A61B 5/0295; A61B 5/00; A61B 5/021

(52) U.S. Cl. CPC A61B 5/0295; A61B 5/7257; A61B 5/02125; A61B 5/021

(57) ABSTRACT

A pulse wave imaging system, more generally a system for measuring blood perfusion, comprising a configuration of a computing device and a camera is described. The system, and the methods used therein, produce a more robust measurement of several tissue health indicators. Variations and improvements on the basic system are also described.

### FIELD

The improvements generally relate to the field of medical devices using pulse wave imaging, and more generally measurement of blood perfusion. This approach can allow for remote or wireless measurements.

### BACKGROUND

Blood perfusion is the local fluid or blood flow through the capillary network and extracellular spaces of living tissue. It is generally characterized as the volumetric flow rate per volume of tissue.

A ‘pulse wave’ occurs during the contraction of the left ventricle of the heart; as blood is released from the left ventricle into the systemic circulatory system, pressure increases inside the aorta. This pressure causes slight distension in the elastic arteries and radiates distally throughout the arteries and arterioles.

A pulse wave can be measured using photoplethysmography or PPG, the measure of the volume of an organ.

Usually PPG works by measuring the absorption of light through tissue, or by measuring reflection from tissue. In a typical PPG scenario, a light source and sensor are placed on the skin and record the reflectance or transmission of the light signal. Common heart rate monitors use transmission PPG to measure heart rate through the tip of the finger, earlobe, septum of the nose, or any other thin part of the body where the light will readily pass through. Reflectance PPG can, however, be measured anywhere on the skin.

However, PPG can also be measured by displacement of the skin caused by a pulse wave.

Remote PPG (rPPG) is a single point measurement method, which captures reflectance PPG remotely—i.e. without touching the skin.

Several fluorescence-based techniques have been used to assess tissue perfusion: fluorescein angiography and indocyanine green (ICG) fluorescent angiography. However, these methods require dye injection, which is not recommended in some cases.

Existing research into reflectance-based PPG has been unsatisfactory from a robustness points of view, in that the existing literature has results that are not robust in that they do not give consistent and/or accurate results when encountering variations in the characteristics of the skin and/or person being examined, and differences in the measurement conditions, such as distance of the camera from the subject, differences in lighting, or uncontrolled movement of the subject being measured and/or the camera.

Improvements in PPG and rPPG are important for addressing several matters, such as skin flap viability. Flap viability is an important problem in reconstructive surgery. Blood perfusion can be a helpful indicator of flap viability. It has been found that perfusion score had a positive predictive value of removing nonviable skin of 88 percent and a negative predictive value of removing healthy skin of 16 percent in patients with mastectomy.

It is therefore desirable to have systems and methods of measuring PPG through reflectance that are more robust than current methods and systems and can be used for remote measurement.

It also well-known that it is preferrable to collect more quality data for analysis (although burying quality data in low-quality data or noise can cause problems), and that it is preferrable to use better equipment, specifically cameras. However, there is little knowledge of under what conditions less expensive equipment (specifically cameras) can be used and still generate a robust result, how much we can reduce the amount of data collected, and what additional equipment or methods can increase the quality of the data collected. These are key practical issues to the development of systems and methods of pulse wave imaging for widespread use and would be desirable to have.

### SUMMARY

A system, devices and method for using the device are described that allow improved measurement of blood perfusion using reflectance PPG, and in particular do not require the use of dyes or invasive techniques. This method can also allow remote measurement. This is useful for several purposes including assessing blood pressure, pulse transit time, blood pressure time waveform, jugular venous pulse monitoring, and flap viability assessment. The system and methods described herein provide for a more robust measurement.

Using one or more cameras, changes in the blood volume in a tissue can be detected through measuring reflectance, which are then used to extract information about, for example, pulse wave velocity, pulse transit time, and blood perfusion.

The inventors have investigated what equipment and methods can be used to obtain robust results, and have specifically investigated these for use in practical, remote applications. There is an unpredictable, difficult relationship between factors such as the purpose of the analysis, illumination of the target area, channels or filters on the camera, the method of analysis, use of optical clearing agents, distance from the camera to the target area and control of that distance, and the quality of the camera and the amount of data collected to provide a robust result.

Experiments have been performed to identify the device and method parameters described herein. These have shown that for some purposes, by capturing and processing reflectance data over an area rather than a single or a few discrete points, a more robust analysis of blood perfusion can be achieved. These experiments have also shown that for other purposes data often has to be captured at a high frame rate, and for good results the data needs to be captured for separate segments of the tissue and then filtered.

The system can be extended through the use of multiple cameras, and through the use of illumination equipment. By choosing the appropriate illumination, better sets of data and more robust results can be derived from the images of the tissue volume over time.

This is useful for several purposes including assessing blood pressure, pulse transit time, blood pressure time waveform, jugular venous pulse monitoring and flap viability assessment.

The quality of the measurement is dependent upon surrounding conditions, such as the illumination, the distance from the camera to the target area and whether the distance remains constant or varies (which can range from the unsteadiness associated with holding a camera in place to movements of the person being analyzed) or the condition of the target's skin. The inventors have identified the minimum requirements to achieve robust measurement under good or ideal conditions but recognize that if conditions are not ideal or good, more aggressive techniques may be needed. For example, in some cases as listed below a 20 frames per second recording may work in ideal or good conditions: however, to get a robust result (i.e. a result that will be reliable despite less than ideal conditions) it may be necessary to capture video at more than 120 fps. In light of this, certain changes in equipment and technique are described that can generally be used to increase the robustness of the measurement, which include changes in illumination, the use of different channels from the camera in the measurement, the frames per second speed of the camera, and the use of optical clarifiers.

Optionally, the system can be set up to work wirelessly or by cable, and can include remote sensing (i.e. that the camera and optional illuminator are physically distant from the data storage and processing units).

In accordance with one embodiment of the present invention, there is provided a system for measuring changes in the blood volume in a tissue (plethysmography) comprising: A camera, the camera in communication with a non-transitory computer-readable memory and a processor, and the memory and processor being in communication with a display device, the camera being located to be able to record reflectance images of a target skin area; wherein the camera, processor, memory and display device are configured so that: the camera records a series of equally spaced in time reflectance images of a target skin area; the reflectance images are segmented by the processor into several non-overlapping spatial segments, each segment comprising an array of pixels; the reflectance for each spatial segment over the pixels in the segment is averaged for each point in time; the time series of the average reflectance for each spatial segment is used to extract a set of PPG signals; the PPG signals are processed for to extract useful information about blood flow in tissues; and the useful tissue information is communicated to a user.

In an aspect of this invention, the camera is remote from the non-transitory computer-readable memory and a processor.

In another aspect of this invention, the useful tissue information is jugular venous pulse monitoring, further comprising: the camera recording at least 6 seconds of video having at least one channel of the reflectance of the right or left side of the neck from the sternum to the earlobe at at least 20 frames per second; segmenting the video into blocks of pixels; averaging the reflectance signal for each channel in the video; the step of processing the PPG signals comprises: applying a Fourier Transform or Fast Fourier Transform to the extracted average PPG signals, and using the Fourier Transform coefficients to calculate a pulsation indicia for each segment, and using the location of the transition between segments with high and low pulsation indicia to determine the jugular venous pulse.

In another aspect of this invention, the pulsation indicia is calculated as the ratio of the sum of Fourier Transform coefficients corresponding to 0.5-3 Hz to the zeroth Fourier Transform coefficient.

In another aspect of this invention, the pulsation indicia is calculated as the ratio of the sum of Fourier Transform coefficients corresponding to 0.5-3 Hz to the sum of Fourier Transform coefficients corresponding to 4-8 Hz.

In another aspect of this invention, the block of pixels has a maximum N×N size determined by dividing the vertical field of view of the video by the number of pixels in the vertical field of view to obtain the vertical field per pixel, and then setting the maximum block size to the largest number of pixels that will not exceed the desired accuracy.

In another aspect of this invention, the camera is an RGB or RGB-NIR camera and the output of the red (R) channel is used to extract the PPG signal.

In another aspect of this invention, the video is recorded with a red bandpass filter in the 660-760 nm range and the output is used to extract the PPG signal.

In another aspect of this invention, the camera is an RGB-NIR camera and the output of the red (R), green (G), blue (B) channels is used to extract the PPG signal.

In another aspect of this invention, the gain of the red channel is increased.

In another aspect of this invention, the target skin is illuminated with light in the 660-760 nm wavelengths.

In another aspect of this invention, the system further comprises a means to measure distance from the target area to the camera, recording is automatically initiated once the target area is a pre-determined distance from the camera.

In another aspect of this invention, the means to measure distance from the target area to the camera is a reference object.

In another aspect of this invention, the pre-determined distance is selected to increase the robustness of the measurement of the useful information.

In another aspect of this invention, an optical clearing agent is applied to the skin before recording of the images.

In another aspect of this invention, image registration is used to remove motion artifacts received from the camera.

In another aspect of this invention, the determination of jugular venous pulse is made on a continuous basis, and the system automatically makes adjustments to improve the accuracy and repeatability of the determination of the jugular venous pulse by the system, and indicates both the jugular venous pulse measurement and a measure of its reliability to the user.

In another aspect of this invention, the useful tissue information is pulse wave velocity measurements, and the camera recording at least 10 seconds of video at at least 1000 frames per second; segmenting the video into at least two linearly arranged non-overlapping segments; averaging the reflectance signal for each channel in the video; the step of processing the PPG signals comprises: applying smoothing filters; applying moving average filters, detrending the data, and cross-correlating the PPG measurements to find the time delay between each segment, and using the time delay to calculate the pulse transit time; and the pulse transit time is used to calculate the wave velocity.

In another aspect of this invention, the camera uses a rolling shutter and a frame rate of at least 20 fps.

In another aspect of this invention, the useful tissue information is remote blood pressure assessment comprising: the camera recording at least 10 seconds of video at at least 1000 frames per second; segmenting the video into two non-overlapping segments; averaging the reflectance signal for each channel in the video; the step of processing the PPG signals comprises: applying smoothing filters; applying moving average filters, detrending the data, and cross-correlating the PPG measurements to find the time delay between each segment, and using the time delay to calculate the mean arterial pressure.

In another aspect of this invention, linear regression is used to obtain the relationship between the delay between each segment and the mean arterial pressure.

In another aspect of this invention, a Hilbert transformation is used to obtain the mean arterial pressure from the delays between the segments.

In another aspect of this invention, a lookup table is used to obtain the mean arterial pressure from the delays between the segments.

In another aspect of this invention, the useful tissue information is tissue viability assessment, further comprising: the camera recording at least 10 seconds of video at least 20 frames per second; segmenting the video into N×M blocks of pixels; averaging the reflectance signal for each channel in the video; the step of processing the PPG signals comprises: applying a Fourier Transform to the extracted average PPG signals, and using the Fourier Transform coefficients to calculate a tissue viability indicia for each segment.

In another aspect of this invention, the tissue viability indicia is calculated as the ratio of the sum of Fourier Transform coefficients corresponding to 0.5-3 Hz to the zeroth Fourier Transform coefficient.

In another aspect of this invention, the pulsation indicia is calculated as the ratio of the sum of Fourier Transform coefficients corresponding to 0.5-3 Hz to the sum of Fourier Transform coefficients corresponding to 4-8 Hz.

In another aspect of this invention, the block of pixels has a maximum N×N size determined by dividing the vertical field of view of the video by the number of pixels in the vertical field of view to obtain the vertical field per pixel, and then setting the maximum block size to the largest number of pixels that will not exceed the desired accuracy.

In another aspect of this invention, the camera is an RGB camera and the output of the green (G) channel is used to extract the PPG signal.

In another aspect of this invention, the video is recorded with a green bandpass filter and the output is used to extract the PPG signal.

In another aspect of this invention, the camera is an RGB or RGB-NIR camera and the output of the red channel minus the green channel (R-G) is used to extract the PPG signal.

In another aspect of this invention, the camera is an RGB-NIR camera and the output of the red (R), green (G), blue (B) channels is used to extract the PPG signal.

In another aspect of this invention, the gain of the green channel is increased.

In another aspect of this invention, the target skin is illuminated with light in the 540-570 nm wavelengths.

In another aspect of this invention, the system further comprises a means to measure distance from the target area to the camera, recording is automatically initiated once the target area is a pre-determined distance from the camera.

In another aspect of this invention, the means to measure distance from the target area to the camera is a reference object.

In another aspect of this invention, the pre-determined distance is selected to increase the robustness of the measurement of the useful information.

In another aspect of this invention, an optical clearing agent is applied to the skin before recording of the images.

In another aspect of this invention, where image registration is used to remove motion artifacts received from the camera.

In another aspect of this invention, the specular reflection is used to visualize skin displacement caused by pulse wave propagation through blood vessels.

In another aspect of this invention, the target skin area is pre-treated with a substance that increases the specular reflection of the skin. In another aspect of this invention, the substance that increases the specular reflection of the skin is one of glycerin, Vaseline, and baby oil.

### BRIEF DESCRIPTION OF THE DRAWINGS

Embodiments will now be described, by way of example only, with reference to the attached figures, wherein in the figures;

FIG. 1 depicts the overall system;

FIG. 2 depicts a flowchart of an analysis procedure;

FIGS. 3a and 3b illustrates measurements of the skin displacement using reflected light;

FIGS. 4a, 4b, 4c and 4d illustrate measurements on a subject wrist using a rolling shutter camera;

FIG. 5 is an example visualization of tissue viability indicia;

FIG. 6 illustrates imaging tissue perfusion using reference objects;

FIG. 7 illustrates use of image registration; and

FIG. 8 illustrates a flowchart for Continuous Measurement and Dynamic Adjustment.

### DETAILED DESCRIPTION

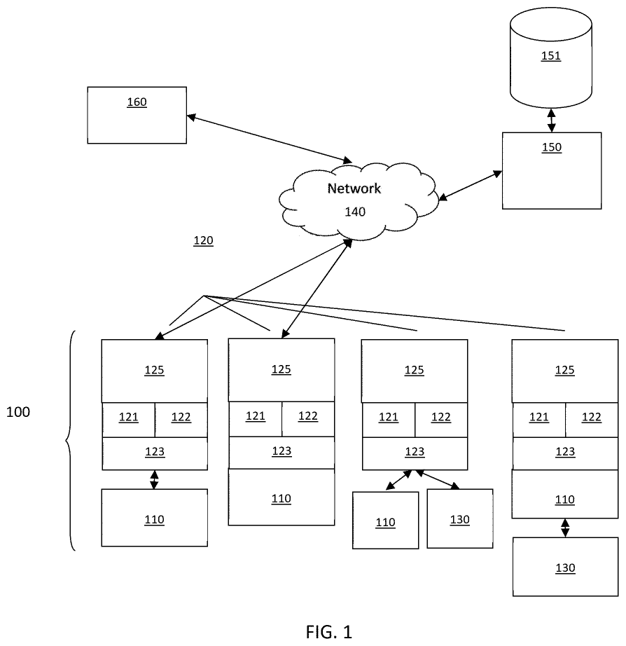

FIG. 1 depicts a view of an example of the overall system architecture, including alternative embodiments. Turning to FIG. 1, the apparatus 100 includes a camera 110. A computing device 120 includes a processor 121, non-transitory computer-readable storage medium or memory 122, and interface unit 123 (that interfaces between the camera and the processor 121 and memory 122. Memory 122 includes executable computer instructions constituting a data processing app 125.

In some embodiments, there is an illumination unit 130. Examples of illumination units are discussed below.

In some embodiments, there is more than one camera 110.

Computing device 120 will usually be a custom-built device built to implement this invention. The computing device 120 may in some cases as set out below be a mobile device, smartphone, tablet, laptop, or a desktop computer. The computing device 120 may be connected to the camera 110 wirelessly or through a cable. In embodiments with an illumination device 130, The computing device 120 may be connected to the illumination device 130 wirelessly or through a cable.

In some embodiments, computing device 120 has sufficient memory and processing speed to store and process data to provide useful results (as discussed below). In other embodiments, data can be stored and/or processed externally in an external database 152 or external processor 150. Communications between computing device 120 and external database 152 and/or external processor 150 may be wireless or cabled communication. These communications may go through a network 140.

In some embodiments, computing device 120 may include a display 124. In some embodiments, a separate display 160 is in communication with either computing device 120 or external processor 150.

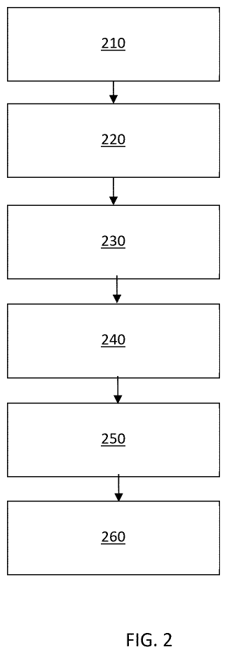

A high-level description of a method for using the apparatus described above to analyze blood perfusion using reflectance PPG is illustrated in FIG. 2.

Turning to FIG. 2, in step 210 at least one camera 110 is used to record a series of equally spaced in time reflectance images of a target skin area. In a preferred embodiment, the camera takes a video of the target skin area.

In step 220, the images of the target skin area are segmented into several non-overlapping spatial segments. Each segment consists of an array of pixels.

In step 230, the reflectance for each spatial segment over the pixels in the segment is averaged for each point in time. (as described below, this step may be expanded to allow for the camera recording the images in two or more channels).

In step 240, the time series of the average reflectance for each spatial segment over the pixels in the segment is used to extract the PPG signal, using some or all of the segments.

In step 250, the PPG signals from step 240, are processed for each segment to extract useful tissue information about blood flow. The exact processing and information extracted depends upon the purpose of the measurement.

In step 260, the results are visualized.

The method and apparatus as described above can determine jugular vein pressure, pulse wave velocity, pulse transit time (PTT) and perform plethysmographic waveform analysis for blood volume in tissue. This can have many uses, including remote blood pressure assessment and tissue viability assessment. As discussed below, the specific steps may be implemented differently depending upon the purpose of the measurement, including changes to the camera as well as changes to the processing.

### Example 1: Jugular Venous Pulse Monitoring

Cardiovascular disease is a leading cause of death worldwide and is responsible for almost ⅓ of all deaths. Thus, early stage screening can significantly improve quality of patient's life and clinical outcome.

Elevated venous pressure is an indicator of various cardiovascular diseases. However, current methods of venous pressure assessment are highly invasive. They require catheterization (surgically inserting a central line into the jugular vein, superior vena cava, or right atrium). Thus, noninvasive methods of central venous pressure (CVP) assessment are of great importance. Catheterization requires surgically inserting a central line into the jugular vein, superior vena cava, or right atrium. This is an invasive procedure requiring surgical expertise. Complications include pain at the cannulation site, local hematoma, infection (both at the site as well as bacteremia), misplacement into another vessel (possibly causing arterial puncture or cannulation), vessel laceration or dissection, air embolism, thrombosis, and pneumothorax requiring a possible chest tube.

The jugular vein (JV) is a major venous extension of the heart's right atrium. Jugular Venous Pulse (JVP) is defined as the oscillating top of vertical column of blood in JV that reflects changes in the right atrium during cardiac cycle. Jugular Venous Pulse can be used to assess Jugular venous pressure and CVP.

Therefore, although the jugular venous pulse (JVP) can provide important clinical insights, JVP examination using catheterization is not routine and reserved primarily for emergent indications. Additionally, since catheter monitoring is limited to measuring a single location, spatial flow perfusion characteristics cannot be assessed, which may encode important clinical information. A noninvasive method of robustly measuring the JVP is desirable.

It is known to measure the JVP using a ruler. A doctor places a patient in a reclined position) (30-45°), puts the base of the ruler at the sternal angle, and measure the height of the JVP visually. Then, CVP (in cm of water) can be calculated as CVP=5 cm+JVP. Here, 5 cm is the known distance between the sternal angle and right atrium. Positive JVP>3 cm above the sternal angle, or sustained JVP>4 cm with abdominal compression, suggests a 3-4×increase in the likelihood of CVP being elevated. However, this approach does not give precise information, and its robustness depends heavily on the skill of the doctor.

The automated detection of the JVP can provide information about central venous pressure noninvasively. The segmented PPG signals measured by this system can detect JVP and provide information about CVP, including waveform information.

The systems and methods described above in relation to FIGS. 1 and 2 can be used to robustly measure JVP.

In a preferred embodiment, the following method is used to robustly assess jugular venous pulse and central venous pressure:

Put a patient in a reclined position with 30-45 degrees tilt.

Step 210: Capture a 10 second video of the reflectance of a target area of skin—preferably the right side of the neck from the sternum to the earlobe, but the process will work with the left side—at 30 frames per second.

Step 220: Segment each frame into n×m blocks of pixels.

Step 230: Average the reflectance signal for each channel for each segment over the pixels. Note that the use of multiple channels is discussed below.

Step 240: Extract average PPG signal for each segment. (for example, from the red (R) channel of the camera)

Step 250: Process the extracted PPG signals. In a preferred embodiment, a Fourier Transform-based periodogram (spectral density) is applied to the time series from step 240, and then the pulsation indicia for each segment is calculated for each segment (e.g. the relative spectral power for pulse range region (0.5-3 Hz). In a preferred embodiment, the following ratio was used as a pulsation indicia: the ratio of the sum of FT coefficients corresponding to 0.5-3 Hz to zeroth FT coefficient. In another embodiment, the following ratio was used as a pulsation indicia: the ratio of the sum of FT coefficients corresponding to 0.5-3 Hz to the sum of FT coefficients corresponding to 4-8 Hz.

In other embodiments, other ratios can be used to extract pulsation indicia.

In some embodiments, the Fast Fourier Transformation (FFT) is used to obtain FT coefficients.

The JVP can be determined based on the location of the transition between segments with high and low pulsation indicia.

Step 260: Visualize the results in a manner usable by the user. In some embodiments, the pulsation indicia are visualized using false color or gray color representation.

Experiments have shown that a consumer-grade 8-bitRGB camera (for example, a smartphone camera) can be used to perform the above method. In other embodiments, high quality (and cost) types of camera (for example a 12-bit RGB camera) can be used.

The two primary factors, which can impact the quality of the gathered signal data are variations of the ambient illumination and motion artifacts.

To minimize the impact of the ambient light, the frame rate can be selected as F=2*f/k, where k is an integer, and f is the utility frequency for a particular country in Hz (e.g., 60 Hz for North America, 50 Hz for Europe). Thus, the framerate can be set to 30, 24, 20, 15, 12, or 10 fps for North America and 25, 20, or 10 fps for Europe. The frame rate of 20 fps can be an example of selection, as it can work without any configurations with external light sources connected to any grid (50 Hz or 60 Hz).

If the ambient light does not flicker with the utility rate (e.g. in the presence of natural light or LED light) or if the ambient light effects are compensated in any other way, then other frame rates can also be used.

To minimize motion artifacts, In the preferred embodiment, the camera position is stabilized.

If physical stabilization is not feasible then the stabilization can be achieved during image processing. In some embodiments, image registration is used. Image registration is discussed in further detail below.

In another embodiment, the motion artifact frequency can be detected by other means (e.g. built-in accelerometer) and the motion artifact frequency excluded during processing in step 250.

In one embodiment, the n×m blocks of pixels are a maximum block size N×N that can be determined based on the required accuracy. For example, if the vertical field of view is 20 cm and 640×480 pixel video is used, then, the geometric size per pixel is 20 cm/640 pixels =0.031 cm/pixel. Thus, if 0.5 cm accuracy is required for JVP determination, then the block size should not exceed the required accuracy (0.5 cm/(0.031 cm/pixel)=16.12 pixels) and should be 16×16 or less.

As discussed above, a camera in a smartphone can be used for this purpose. In such cases, the other steps of the method (steps 220-250) can be performed by a modern smartphone, including visualizing the results on the screen of the smartphone.

In some embodiments, 384×288 video mode is used. In other embodiments, 640×480 video is used. In a preferred embodiment, the number of frames in the PPG series is 2ⁿ, where n is an integer, since this allows for faster Fast Fourier Transform processing. In an example embodiment, 256, 512, or 1024 images are used to extract the pulsation indicia.

In a preferred embodiment, illumination and/or the camera channels are used to improve the systems and methods.

The faint jugular vein (“JV”) signal is masked by the dominant carotid artery (“CA”) signal. They have the same frequency, so frequency- based-filtering is ineffective. In the preferred embodiment, the optical properties of oxy-(HbO2) and deoxy-(RHb) hemoglobins are used to resolve arterial and venous pulsing components. It is well known that the arterial blood is highly oxygenated with SO2(a measure of oxygenation in the blood) in the range of 97-99%. The venous blood has significantly lower oxygen saturation, which can be in 40-60% range. Thus, in order to increase sensitivity to the venous vascular component the difference in absorption spectra of these blood components can be used. In particular, in the 660-760 nm range have approximately 3-10×higher absorption of RHb compared to HbO2, which will significantly increase the contrast ratio.

In one embodiment, an RGB camera (i.e. a camera that measures red, green and blue light separately and simultaneously in different channels) is used to record the reflectance signal and the output of the red channel (R) of the camera is used to extract the PPG. In another embodiment, an RGB-NIR camera (i.e. a camera that measures red, green, blue and near-infrared separately and simultaneously) is used to measure the reflectance signal, and the red channel (R) of the camera is used to extract the PPG. In another embodiment, a monochrome camera in combination with a red bandpass filter is used to extract the PPG signal. The filter should allow the passage of light in the red range of the spectrum.

In other embodiments, an RGB camera or an RGB-NIR camera is used, and the red channel (R) minus the green channel (R-G) is used to extract the PPG signal. Such subtraction compensates for the changes in ambient light.

In other embodiments, an RGB-NIR camera is used, and the NIR channel (NIR) minus the red channel (R) (NIR-R) is used to extract the PPG signal. Such subtraction compensates for the changes in ambient light.

In other embodiments, an RGB camera or an RGB-NIR camera is used, and the output of all three channels (red (R), green (G), and blue (B)) or four channels (red (R), green (G), blue (B), and near infrared (NIR)) are used to extract the PPG signal.

In some embodiments, independent component analysis (ICA), principal component analysis (PCA) or a similar technique is used to unmix the PPG signal.

In a further alternative embodiment, to increase the dynamic range of the signal and improve signal-to-noise ratio, either: a) the gain of the red channel is increased, or b) the target are can be illuminated with additional light in the 660-760 nm range of the spectrum, or both approaches can be implemented.

Generally, ambient light is used to illuminate the target area. However, to increase contrast and allow for better sensing/data collection, additional light sources may be used to illuminate the target area.

In one embodiment, a narrowband light source with a central wavelength in the 660-760 nm range is used to illuminate the target area. In another embodiment, a light emitting diode (LED) with a central wavelength in the 660-760 nm range is used to illuminate the target area.

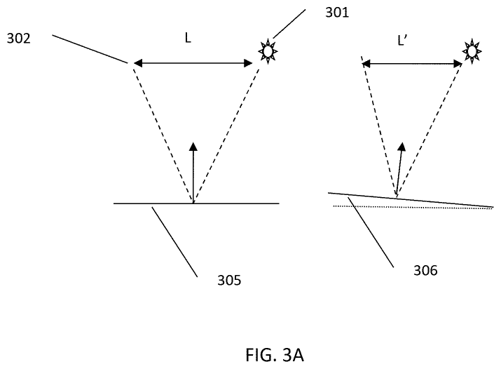

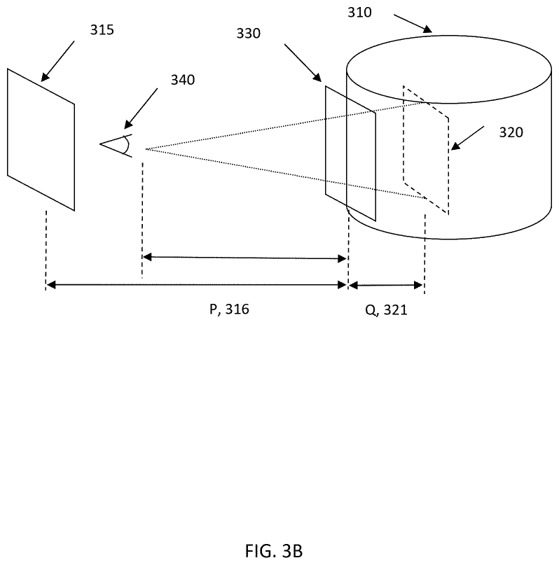

The method can be further improved by detecting changes in geometry of the skin surface. The changes in geometry due to Jugular Venous pulsations lead to changes of the angle between normal vector to the surface and the direction of the incoming light. If the surface reflects the light, then these changes in the angle can be detected (FIGS. 3a and 3b).

Turning to FIG. 3a, for example, 10 μm displacement of the skin is very hard to detect. However, if one edge of the 1 mm piece of skin is displaced by 10 μm from the position 305 into position 306, it will cause the change in the angle between normal vector and direction of light by 0.01 rad. If the surface is illuminated by a point light source 301 located at 1 m from the skin and the image plane 302 is located at the same distance, then the displacement of the reflectance spot on the camera will be ΔL=L−L′=2*0.01*(1+1)=0.04 m, which is very easy to register.

This observation can be turned into an imaging technique, which we termed a specular vascular imaging. Turning to FIG. 3b. For the purposes of specular imaging, a neck can be considered as a cylindrical (convex) mirror, 310 with approximate radius r=6-7 cm (circumference 37.5-44 cm) and the height H=10-15 cm.

If the neck, 310 is illuminated by a rectangular light source, 315 which is located on the distance P, 316 from the neck, then the light source will be visible as an image of the light source, 320 located on the distance Q, 321 inside the neck.

P and Q are connected by the mirror formula (here all distances are positive)

$$\frac{1}{P} - \frac{1}{Q} = - \frac{2}{r} \qquad ( 1 )$$

Thus, the image, 320 of the light source, 315 with height h and width 2w is be a rectangle with the height h and width 2w′, which is located on the distance Q inside the mirror. Here, Q can be found from Eq.1. The semi-width of the image, 320 w′ can be found using the magnification formula for the mirror (m=Q/P):

$${w}^{′} = wm = w \frac{1}{1 + 2 P / r} \qquad ( 2 )$$

For example, for realistic r=6 cm and P=12 cm, Q=2.4 cm and m=0.2. Thus, to image the target area, 330 with the width W=3 cm, the light source with at least 15 cm (W/m) width is required.

Thus, the observer, 340 will see a bright area (a rectangle the height h and width 2w′ for the perfectly cylindrical neck), which is located on the distance Q inside the mirror. Any changes in the light distribution within this bright area visible by an observer 340 will reflect changes in the surface geometry of the reflecting mirror (neck), 310,

In the preferred embodiment the target area is illuminated with a rectangular light source with a height slightly more (e.g. by 20%) than the height of the target area, 330. The rectangular source is aligned with the body part (neck) and the camera is located in the central plane perpendicular to the axis of the light source.

In the exemplary embodiment, the height and width of the rectangular source is in the range of 15-20 and 20-25 cm, respectively.

However, the normal skin does not reflect light in a specular reflection fashion. It still has a specular reflection light component (Fresnel reflection); however, it is scattered across all angles due to roughness of the skin surface. Nevertheless, it is well known that an “oily” or edematic skin (i.e. skin with edema) presents serious specular reflection problems in photography. Thus, to increase specular reflectance, the imaged area can be pre-treated with an oily substance (e.g. glycerin, Vaseline, or Baby Oil), or another substance that causes smoothing of the surface of the skin and increases the specular reflection.

The proposed invention has several significant advantages over existing remote pulse monitoring techniques (PPG): a) The specular reflection depends very slightly on the wavelength and does not depend on chromophores inside the tissue. Thus, it works for any skin tone, including dark skin, where existing PPG methods do not work. b) The specular reflection is quite strong; thus it requires less expensive equipment to achieve a required signal to noise (SNR) ratio.

### Example 2: Pulse Wave Velocity Measurements

The general system and methods discussed above in relation to FIGS. 1 and 2 may be used to measure pulse wave velocity (“PWV”). The speed at which the pulse wave travels through the arteries can reveal key information about the health of the arteries, including elasticity, stiffness, and even potential calcification and stenosis as well as the viscosity of blood.

PWV in peripheral blood vessels is usually in the range of 5-10 m/s. However, it can be higher due to an increase in stiffness of blood vessels (for example, due to calcification). The pulse transit time (“PTT”) between segments of the same skin area depends on the distance across which the pulse is being measured or the field of view (“FOV”). For a 15 cm linear size FOV, the PTT can be anticipated to range between 15-30 ms.

To determine the PWV with the accuracy of 10%, the PTT should be determined with at least the same accuracy. Also, the implementation needs to account for increased PWV in calcified vessels.

Thus, for a 15 cm FOV, it has been determined that an approximately 1000 fps (frames per second) camera with global shutter is required. (A global shutter means that all of the pixels in the image has the same exposure time, or the same starting and ending time during which they are exposed to light).

In a preferred embodiment, the following method is used to assess pulse wave velocity measurements:

Step 210: Capture a 10 second video of the reflectance of a target area of skin at 1000 frames per second.

Step 220: Segment the video from Step 210 into at least two linearly arranged non-overlapping segments.

Step 230: Average the reflectance signal for each channel for each segment over the pixels.

Step 240: Extract average PPG signal from each segment. For example, red channel (R) minus the green channel (R-G) can be used to extract the PPG signal.

Step 250: Process the extracted average PPG signals. In a preferred embodiment, smoothing filters and moving average filters are applied (in a further preferred embodiment, the Savitsky-Golay and boxcar filters are used). The data is then detrended, to remove any changes in the mean of the measurement. Cross-correlate the segmented PPG measurements to find the time delay between each segment, thus measuring the pulse transit time or PPT. The PPT can then be used to calculate the pulse wave velocity.

Step 260: Visualize the results in a manner usable by the user.

Due to the different purpose for this embodiment, the data collection in this method requires video at 1000 fps. As a result, for the purpose of measuring pulse wave velocity, a camera as typically embodied in a smartphone cannot be used, and a high quality camera is needed.

In one embodiment, a 12-bit RGB camera (such as a Basler ac-A2000-165uc, Basler AG, Germany) capturing videos at a high frame rate (around 1000 fps) is used.

The two primary factors, which can impact the quality of the gathered signal is flickering of the ambient illumination and motion artifacts.

To minimize the impact of the ambient light, the utility frequency for a particular country in Hz (e.g., 60 Hz for North America, 50 Hz for Europe) and its harmonics should be filtered out during processing before step 220. It can be done by using a low pass filter with cut-off frequency<100 Hz.

To minimize the impact of low-frequency ambient light artifacts (e.g. emergency vehicle lighting), the high pass filter with cutoff frequency>1 Hz can be used, provided that the patient's heart rate is higher than 60 bps.

If the ambient light does not flicker (e.g. natural light or LED light) or ambient light effects are compensated in any other way, then this step is not required.

To minimize motion artifacts, In the preferred embodiment, the camera position is stabilized.

If physical stabilization is not feasible then the stabilization can be achieved during image processing. In some embodiments, image registration is used. Image registration is discussed below.

In other embodiment, the motion artifact frequency can be detected by other means (e.g. built-in accelerometer) and the motion artifact frequency excluded during processing during step 250 from consideration.

In another option, a camera with a rolling shutter can be used. In this case, a smartphone camera with the normal frame rates (e.g. 20-30 fps) can be used.

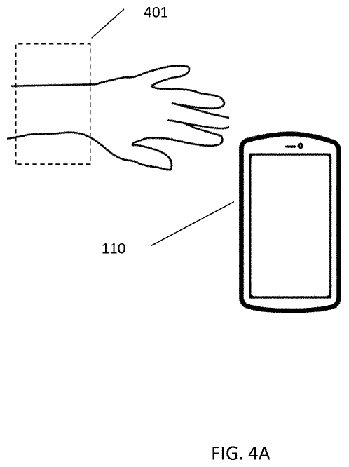

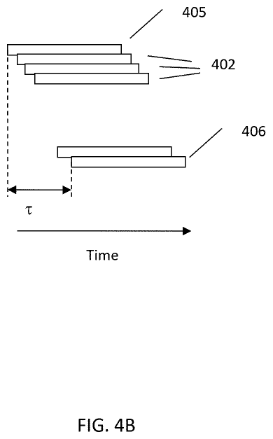

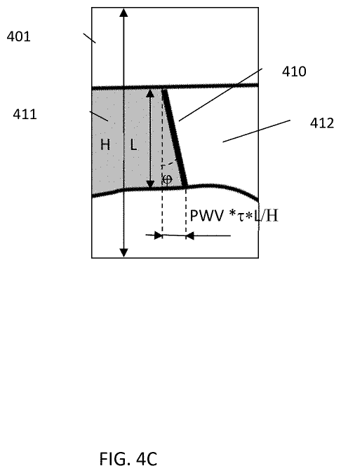

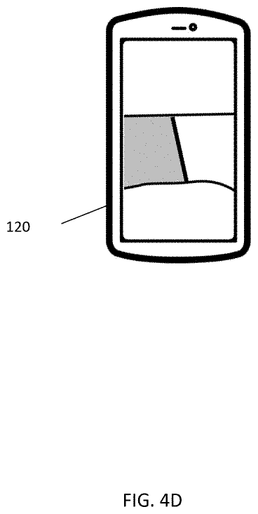

FIGS. 4a to 4d illustrate measurements using a rolling shutter camera according to the above embodiment. Turning to FIG. 4a, a field of vision of FOV 401 is chosen (in the case of the illustration in FIG. 4a the FOV 401 corresponds to an image taken by a smart phone camera 110 with a rolling shutter and includes the back of a wrist). Turning to FIG. 4b, as this camera has a rolling shutter, the different rows of pixels 402, are exposed to light at different times. For example, as seen in FIG. 4b the exposure time of the first-row 405 and the last row 406 will be shifted by T, (equivalently, row 405 and row 406 are exposed to light for the same amount of time but row 406's exposure starts T later than row 405), and T is typically in the range of several milliseconds.

Turning to FIG. 4c, it can be seen that the FOV 401 includes a vertical dimension H and the vertical dimension of the tissue to be measured L.

The rows 405 through 406 should cover the range H.

In the example illustrated in FIGS. 4a-4d, the camera rows 405 through 406 are approximately aligned with arterial direction of a body part under consideration—the top of the wrist.

The system should be optimized to detect the gradual changes in reflectance row-by-row. For example if the pulse wave propagation with PWV=10 m/s is captured using the rolling shutter camera with 1000 rows and τ=6 ms delay in exposure between the first and the last row, and the wavefront 410 between areas with higher absorption (HAA) 411, and lower absorption (LAA), 412, is captured in the first row, then the wavefront, 410 in the last row (measured with a τ=6 ms delay compared to the first row) will be shifted by 6 cm, and this should be captured within the FOV

The PWV can be found from the angle of the wavefront propagation φ: tan(φ)=PWV*τ/H, where H is the vertical dimension of the FOV, 401.

Optionally, edge detection algorithms can be used to detect the wavefront.

In another embodiment, wavefront detection is further optimized by selecting only frames from the video that are close to the systolic peak. For example, frames can be selected by extracting the average PPG signal from the video and selecting frames with the highest absorption in the green channel.

### Example 3: Remote Blood Pressure Assessment

The general system and methods discussed above in relation to FIGS. 1 and 2 may be used to remotely measure blood pressure. Remote blood pressure assessment is based on remote measurement of pulse transit time (PTT) between two segments with a known distance between them.

In a preferred embodiment, the following method is used to assess blood pressure remotely:

Step 210: Capture a 10 second video of the reflectance of a target area of skin at 1000 frames per second. For remote measurement, the camera capturing the video can be wirelessly connected to the display and/or processing hardware.

Step 220: Select two non-overlapping segments of the target area (for example, if the target area is the face, select cheeks and forehead).

Step 230: Average the signal for each channel for each segment.

Step 240: Extract average PPG signal from each segment. For example, red channel (R) minus the green channel (R-G) can be used to extract the PPG signal

Step 250: Process the extracted average PPG signals. In a preferred embodiment, smoothing filters and moving average filters are applied (in a further preferred embodiment, the Savitsky-Golay and boxcar filters are used). The data is then detrended, to remove any changes in the mean of the measurement. Cross-correlate the segmented PPG measurements to find the time delay between each segment, thus measuring the pulse transit time or PPT. The PPT can then be used to reconstruct the mean arterial pressure.

Step 260: Visualize the results in a manner usable by the user.

In one embodiment, a 12-bit RGB camera (Basler ac-A2000-165uc, Basler AG, Germany) capturing videos at a high frame rate (1000 fps) is used.

In one embodiment, linear regression can be used to obtain the relationship between PTT and mean arterial pressure (MAP) (for example, by using least square fitting). Then, mean arterial pressure can be reconstructed from PTT measurement using a linear function:

MAP=a*1/PTT+b where a and b were determined from the least square fitting.

In another embodiment the Hilbert transformation is used to obtain arterial pressure.

In other embodiments, a lookup table can be used to reconstruct mean arterial pressure from PTT. (i.e. a pre-generated table can be used that presents multiple solutions for the direct problem).

The two primary factors, which can impact the quality of the gathered signal is flickering of the ambient illumination and motion artifacts.

To minimize the impact of the ambient light, the utility frequency for a particular country in Hz (e.g., 60 Hz for North America, 50 Hz for Europe) and its harmonics should be filtered out during processing before step 220. It can be done by using a low pass filter with cut-off frequency<100 Hz.

To minimize the impact of low-frequency ambient light artifacts (e.g. emergency vehicle lighting), the high pass filter with cutoff frequency>1 Hz can be used, provided that the patient's heart rate is higher than 60 bps.

If the ambient light does not flicker (e.g. natural light or LED light) or ambient light effects are compensated in any other way, then this step is not required.

To minimize motion artifacts, In the preferred embodiment, the camera position is stabilized.

If physical stabilization is not feasible then the stabilization can be achieved during image processing. In some embodiments, image registration is used. Image registration is discussed below.

In other embodiment, the motion artifact frequency can be detected by other means (e.g. built-in accelerometer) and the motion artifact frequency excluded during processing step 250 from consideration.

The method can be further improved, by combining it with electrocardiography (ECG) signal collection. In this case, the pulse transit time, PTT is measured as a time delay between the R peak of the ECG signal and systolic hollow of the PPG signal.

### Example 4: Tissue Viability Assessment

Flap viability assessment during reconstructive surgery is of great importance. For example, the incidence of mastectomy skin necrosis is substantial, ranging from 10 to 30 percent.

Blood perfusion can be a helpful indicator of flap viability. It has been found that perfusion score had a positive predictive value of removing nonviable skin of 88 percent and a negative predictive value of removing healthy skin of 16 percent in patients with mastectomy.

The general system and methods discussed above in relation to FIGS. 1 and 2 may be used to remotely measure blood perfusion, and therefore tissue viability assessment. The perfusion index can be linked to the amplitude of the PPG signal. Thus, the segmented PPG signals measured by this system can provide information about perfusion in various parts of the flap.

In a preferred embodiment, the following method is used to assess tissue viability:

Step 210: Capture a 10 second video of the reflectance of a target area of skin at 30 frames per second.

Step 220: Segment the video from Step 210 into n×m blocks of pixels.

Step 230: Average the signal for each channel for each segment.

Step 240: Extract average PPG signal from each segment. For example, red channel (R) minus the green channel (R-G) can be used to extract the PPG signal

Step 250: Process the extracted average PPG signals. In a preferred embodiment, a Fourier transform-based periodogram (spectral density) is applied to the data from Step 240, and then tissue viability indicia is calculated for each segment. In some embodiments, the following ratio was used as a tissue viability indicia: the ratio of the sum of FT coefficients corresponding to 0.5-3 Hz to zeroth FT coefficient. In another embodiment, the following ratio was used as a tissue viability indicia: the ratio of the sum of FT coefficients corresponding to 0.5-3 Hz to the sum of FT coefficients corresponding to 4-8 Hz.

In other embodiments, other ratios can be used to extract pulsation indicia. In some embodiments, the Fast Fourier Transform (FFT) is used to obtain FT coefficients.

Step 260: Visualize the results in a manner usable by the user.

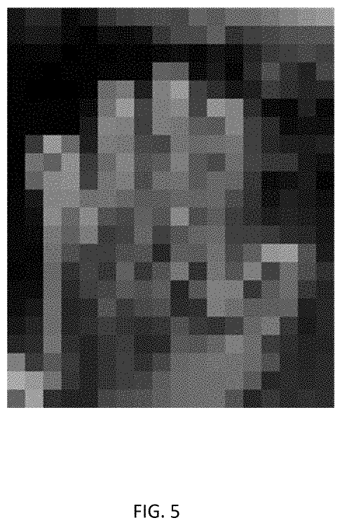

In some embodiments, the tissue viability indicia are visualized using false color or gray color representation. FIG. 5 depicts a view of the visualization of tissue viability indicia with grey color representation according to some embodiments. In some embodiments, perfusion and other tissue health indicia can be displayed on a screen.

In other embodiments, perfusion and other tissue health indicia can be projected on the tissue to guide surgical intervention.

Experiments have shown that a consumer-grade 8-bit RGB camera (for example, a smartphone camera) can be used to perform the above method. The example of the tissue health indicia visualization captured by a consumer grade camera is presented in FIG. 5. In other embodiments, high quality (and cost) types of camera (for example a 12-bit RGB camera) can be used.

In one embodiment, the n×m blocks of pixels are a maximum block size N×N that can be determined by the required accuracy. For example, if the vertical field of view is 20 cm and 640×480 pixel video is used, then, the geometric size per pixel is 20 cm/640=0.031 cm. Thus, if 0.5 cm accuracy is required for tissue viability determination, then the block size should not exceed the required accuracy and should be 16×16 or less. In some embodiments, the block size is 16×16 pixels. In another embodiment, the block size is 20×20 pixels, but not less than 15×15 pixels.

As discussed above, a camera in a smartphone can be used for this purpose. In such cases, the other steps of the method (steps 220-250) can be performed by a modern smartphone, including visualizing the results on the screen of the smartphone. In some embodiments, 384×288 video mode is used. In other embodiments, 640×480 video is used. Other video formats can be used.

The two primary factors, which can impact the quality of the gathered signal is variations of the ambient illumination and motion artifacts.

To minimize the impact of the ambient light, the frame rate can be selected as F=2*f/k, where k is an integer, f is the utility frequency for a particular country in Hz (e.g., 60 Hz for North America, 50 Hz for Europe). Thus, the framerate can be set to 30, 24, 20, 15, 12, and 10 fps for North America and 25, 20, and 10 fps for Europe. The frame rate of 20 fps can be an example of selection. It can work without any configurations with external light sources connected to any grid (50 Hz or 60 Hz).

If the ambient light does not flicker with the utility rate (e.g. natural light or LED light) or ambient light effects are compensated in any other way, then other frame rates can also be used.

To minimize motion artifacts, In the preferred embodiment, the camera position is stabilized.

If physical stabilization is not feasible then the stabilization can be achieved during image processing. In some embodiments, image registration is used. Image registration is discussed below.

In other embodiments, the motion artifact frequency can be detected by other means (e.g. built-in accelerometer) and the motion artifact frequency excluded during processing step 250 from consideration.

In some embodiments, 384×288 video mode is used. In other embodiments, 640×480 video is used. In an example embodiment, the number of frames in the PPG series is 2ⁿ, where n is an integer, as such data can be more quickly processed through a Fast Fourier Transform. In an example embodiment, 256, 512, or 1024 images are used to extract tissue viability indicia.

In a preferred embodiment, illumination and/or the camera channels are used to improve the systems and methods.

The green range of the spectrum may be used to better detect changes in oxyhemoglobin distribution, which corresponds to blood distribution. Vascularized tissue expands and contracts in volume by approximately 1-2% with each incoming systolic blood pressure wave. Since the oxygenation of arterial blood is close to 100% (95-99%), the volume changes are primarily associated with oxyhemoglobin. Oxyhemoglobin has absorption maxima in the green range of the spectrum (near 540 and 570 nm).

The method can be improved by using the green channel (G) or the red (R) minus green (G) channel to extract PPG signals.

In one embodiment, an RGB camera (i.e. a camera that measures red, green and blue light separately and simultaneously) is used to record the reflectance signal and one or more reflectance signals are combined to extract the PPG. In one embodiment, the output of the green channel (G) of the camera is used to extract the PPG. In another embodiment, an RGB-NIR camera (i.e. a camera that measures red, green, blue and near-infrared separately and simultaneously) is used to measure the reflectance signal, and the green channel (G) of the camera is used to extract the PPG. In another embodiment, a monochrome camera in combination with a green bandpass filter is used to extract the PPG signal. The filter should allow the passage of light in the green range of the spectrum.

In other embodiments, an RGB camera or an RGB-NIR camera is used, and the red channel (R) minus the green channel (R-G) is used to extract the PPG signal. Such subtraction compensates for the changes in ambient light.

In other embodiments, an RGB camera or an RGB-NIR camera is used, and the output of all three channels (red (R), green (G), and blue (B)) are used to extract the PPG signal. In some embodiments, independent component analysis (ICA), principal component analysis (PCA) or a similar technique is used to derive the PPG signal.

The reflectance of tissue in a green range of the spectrum is quite low (around 20%). In a further alternative embodiment, to increase the dynamic range of the signal and improve signal-to-noise ratio, either: a) the gain of the green channel is increased, or b) the target are can be illuminated with additional light in the green range of the spectrum, or both approaches can be implemented.

Generally, ambient light is used to illuminate the target area. However, to increase contrast and allow for better sensing/data collection, additional light sources may be used to illuminate the target area.

In one embodiment, a narrowband light source with a central wavelength close to an absorption maximum of oxyhemoglobin in the green range of the spectrum (540 nm and 570 nm) is used to eliminate the target area. In another embodiment, a light emitting diode (LED) with a central wavelength close to 540 nm or 570 nm is used to illuminate the target area.

Refinements: Distance

In a further embodiment, to better account for the distance between segments and/or segment size in the analysis, the distance between the camera and the target area can be pre-determined. Control of the distance can allow the predetermined use of optimally (or at least preferably) sized segments, which allows for more robust results. This also allows the system to better account for aspects of the surrounding environment that could affect the measurement. Determination of the distance allows for a more robust measurement of pulse wave velocity and other useful information for the purposes described above.

The camera can be positioned on the predetermined distance by using: a reference object of a known size placed on a target area, or anatomical features, or other means known to persons skilled in the art (for example, using mechanical, optical, or acoustical means).

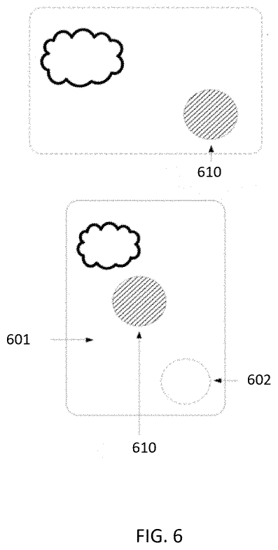

FIG. 6 depicts a view of a schematic of imaging tissue and reference objects. By using reference objects, the distance between the camera and the target area may be measured, either for the purpose of adjusting the camera to be a pre-determined distance from the target area, or for making a measurement of the distance between the camera and the target area, either of which can be used to improve the robustness of the analysis. Turning to FIG. 6, in one embodiment, screen markers 602 displayed on a screen 601 of the computing device 120 (for example, a mobile device 120) can be used to position the camera 110 an optimal distance away from the reference object 610. The camera 110 should be positioned such that screen markers 602 line up with the reference object 610 to ensure an optimal image-capturing distance is achieved.

In an example embodiment, object recognition by data processing app 125 can be used to position the device at the optimal image capturing distance by changing the distance until a selected screen size of the reference object, in pixels, is being captured. In another embodiment, this is performed by the user of the device. In either case, the camera may be activated automatically or by the user.

In some embodiments, the screen size of anatomical features (for example, a nail blade), in pixels, is used to position the device at the certain distance by changing the distance until a selected screen size of the reference object, in pixels, is being captured.

Other distance measuring devices known to persons skilled in the art, such as a rangefinder or ruler, can be used to position the device at the optimal distance.

In some embodiments, the computing device attached to the camera triggers automatic image capturing upon positioning at the proper distance. In an example embodiment, computing device 120 (for example, a mobile device 120) uses object recognition to trigger automatic image capturing once a certain screen size of the reference object, in pixels, is achieved. In other embodiments, image capture is triggered by a user once a certain distance is achieved.

In some embodiments, the distance to the target area is not fixed, but measured using some means. In the exemplary embodiment, it is determined using the reference object 610 of the known size. In this case, the distance can be determined based on the size of the reference object on image in pixels. In the preferred embodiment, the reference object 610 has a circular shape. In other embodiments, other means (ruler, rangefinder, acoustical) are used to determine the distance.

Refinement: Narrow Band Illumination

For all of the methods described above, the data collection can also be improved by narrow-band illumination. In the preferred embodiment, the target area is illuminated by a light in the 660-760 nm range for jugular venous pulse monitoring, or with a central wavelength close to an absorption maximum of oxyhemoglobin in the green or NIR range of the spectrum (540 nm, 570 nm, or 850-950 nm) for other modalities

Refinement: Pulsed Narrow Band Illumination

For all of the methods described above, the data collection can also be improved by pulsed narrow-band illumination. In the preferred embodiment, the target area is illuminated by a pulsed light in the 660-760 nm range for jugular venous pulse monitoring, or with a central wavelength close to an absorption maximum of oxyhemoglobin in the green or NIR range of the spectrum (540 nm, 570 nm, or 850-950 nm) for other modalities

In the preferred embodiment the duration of each flash is 2T, where T=1/fps in milliseconds. The cycle consists of flashes with duration 2T milliseconds each, followed by no lit period 2T milliseconds long.

In another embodiment, the duration of each flash is T, where T=1/fps in milliseconds. The cycle consists of flashes with duration T milliseconds each, followed by no lit period T milliseconds long.

The image set illuminated by a pulsed narrow-band light undergoes pre-processing step, which is performed after the capturing images 210 and before the first step of processing 220.

The preprocessing consists of subtracting consecutive images with no illumination from images with illumination.

Refinement: Control of ambient light

If the narrow band illumination is used, the data collection can also be improved by controlling an ambient light.

In some embodiments, a nontransparent screen is used.

In another embodiment the ambient light is dimmed during data collection

Refinements: Optical Clearing

For all of the methods described above, the data collection can also be improved by optical clearing. Skin type and characteristics vary from person not person, and the use of an appropriate optical clearing agent can result in better data collection. In one embodiment, an optical clearing agent with an index of refraction close to the index of refraction of scatterers inside the skin can be applied to the skin area just before imaging to increase the transparency of the skin. In some embodiments, glycerol, glycerol—water solutions, glucose, propylene glycol, polyethylene glycol, DMSO, sunscreen creams, cosmetic lotions, gels, and certain pharmaceutical products are used as the optical clearing agent.

The optical clearing agent may applied transdermally using aerosol, or may be applied transdermally directly to the skin area. In another embodiment, the optical clearing agent is injected to the skin area using an intradermal injection.

Refinement: Image Registration

Motion artifacts can be caused by the motion of the camera (e.g. handheld scenario) or motion of the target area (e.g breathing movements).

In the preferred embodiment, the camera position is stabilized to minimize effect of the camera motion.

However, if handheld operations are required, other means can be used.

In some embodiments, the device uses image registration to reduce motion artifacts. This can be accomplished through phase correlation or block matching, for example.

Further, in some embodiments, the reference object 610 is used for image registration.

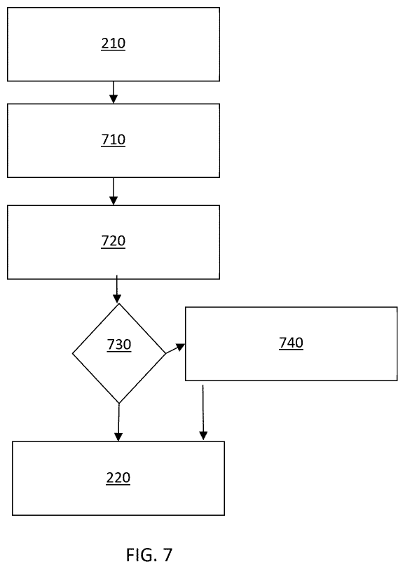

Thus, the following procedure as illustrated in FIG. 7 can be applied to remove motion artifacts (both camera motion and target motion). Turning to FIG. 7:

The process starts after step 210, as described above.

In step 710, several images are being sampled from the dataset collected at step 210. The sampling frequency can be selected based on the potential source of the artifact. If the tremor is suspected (4-9 Hz), then images can be sampled at 2×-3×of the suspected artifact frequency (e.g. every 0.05-0.1 sec), if breathing motion is suspected (0.1 Hz), then images can be sampled every 3-5 sec.

In the step 720, images are compared to detect motion. In some embodiments the phase correlation is used. In other embodiments, block matching is used. In some embodiments, to increase the precision of comparison, a reference object 610 is used.

If motion is detected in step 730, then the additional step of registration (740) is performed before step 220

If no motion is detected on step 730, then the additional step of registration (740) is not required and the algorithm proceed to step 220 directly.

Refinement: Continuous Measurement and Dynamic Adjustment

In a further embodiment, useful information (including blood pressure, pulse transit time, blood pressure time waveform, jugular venous pulse monitoring and flap viability assessment) is continuously measured using continuous video from the camera. Statistical analysis is applied to the continuous stream of results until a statistically robust measurement is achieved. If a robust measurement is not achieved, the system can suggest changes to the user to improve the robustness of the measurement, including changes in distance between the camera and the target, changes in illumination of the target skin area, the application of an optical clearing agent, the use of different channels, and an increase in the frame rate of the camera.

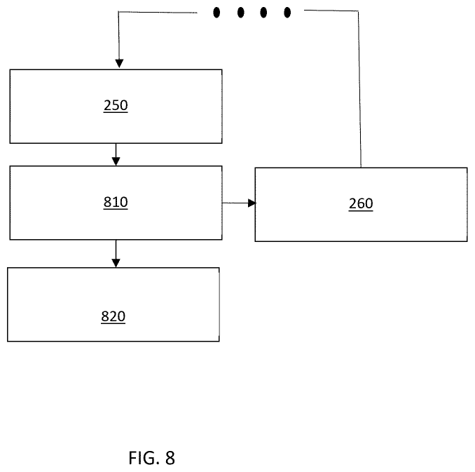

Such a process is illustrated in FIG. 8. Turning to FIG. 8, the outcome of the PPG signal processing 250 is analyzed continuously by a processing block 810. If no pulsation indicia are detected on step 810, or if statistical analysis shows that the pulse indicia are not robust, the system notifies user on step 260 with suggestions to improve the robustness of the reading such as using narrow-band illumination, an optical clearing agent, control of ambient light, or some combination of suggestions. Once a level of robustness is achieved, then the user is informed in step 820. Once a level of robustness is achieved, the user can accept this as a reading and cease the process. However, in the case of continuous operation, processing block 810 will continue to apply statistical analysis to the continuous results from step 250 and if the results become unstable can suggest refinements in step 260.

1. A system for measuring changes in the blood volume in a tissue (plethysmography) comprising:
a camera, the camera in communication with a non-transitory computer-readable memory and a processor, and the memory and processor being in communication with a display device, the camera being located to be able to record reflectance images of a target skin area; wherein the camera, processor, memory and display device are configured so that:
the camera records a series of equally spaced in time reflectance images of a target skin area;
the reflectance images are segmented by the processor into several non-overlapping spatial segments, each segment comprising an array of pixels;
the reflectance for each spatial segment over the pixels in the segment is averaged for each point in time;
the time series of the average reflectance for each spatial segment is used to extract a set of PPG signals;
the PPG signals are processed for to extract useful information about blood flow in tissues; and
the useful tissue information is communicated to a user.

2. The system of claim 1, where the useful tissue information is a pulsation indicia, further comprising:
the camera recording at least 6 seconds of video at at least 20 frames per second;
segmenting the video into blocks of pixels;
averaging the reflectance signal for each channel in the video;
the step of processing the PPG signals comprises: applying a Fourier Transform or Fast Fourier Transform to the extracted average PPG signals, and using the Fourier Transform coefficients to calculate a pulsation indicia for each segment.

3. The system of claim 2, where the pulsation indicia is calculated as the ratio of the sum of Fourier Transform coefficients corresponding to 0.5-3 Hz to the zeroth Fourier Transform coefficient.

4. The system of claim 2, wherein the block of pixels has a maximum N×N size determined by dividing the vertical field of view of the video by the number of pixels in the vertical field of view to obtain the vertical field per pixel, and then setting the maximum block size to the largest number of pixels that will not exceed the desired accuracy.

5. The system of claim 2, where the determination of a pulsation indicia is made on a continuous basis, and the system automatically makes adjustments to improve the accuracy and repeatability of the determination of the pulsation indicia by the system, and indicates both the pulsation indicia measurement and a measure of its reliability to the user.

6. The system of claim 2, where the pulsation indicia is calculated for skin displacement caused by pulse wave propagation through blood vessels measured by specular reflection from the tissue surface.

7. The system of claim 6, where the specular reflection is used for jugular venous pulse monitoring, further comprising:
recording a video having at least one channel of the reflectance of the right or left side of the neck from the sternum to the earlobe; and
using the location of the transition between segments with high and low pulsation indicia to determine the jugular venous pulse and pressure.

8. The system of claim 6, where the target skin area is pre-treated with a substance that increases the specular reflection of the skin.

9. The system of claim 6, where the camera is an RGB or RGB-NIR camera and the output of the any or all channels is used to extract the PPG signal.

10. The system of claim 2, where tissue viability assessment is derived from the pulsation indicia.

11. The system of claim 10, where the camera is an RGB camera and the output of the green (G) channel is used to extract the PPG signal.

12. The system of claim 10, where the target skin is illuminated with light in the 540-570 nm wavelengths.

13. The system of claim 1, where the useful tissue information is pulse wave velocity measurements, and
the camera recording at least 10 seconds of video at at least 1000 frames per second;
segmenting the video into at least two linearly arranged non-overlapping segments;
averaging the reflectance signal for each channel in the video;
the step of processing the PPG signals comprises: applying smoothing filters; applying moving average filters, detrending the data, and cross-correlating the PPG measurements to find the time delay between each segment, and using the time delay to calculate the pulse transit time; and the pulse transit time is used to calculate the wave velocity.

14. The system of claim 13, where the camera uses a rolling shutter and a frame rate of at least 20 fps.

15. The system of claim 1, where the useful tissue information is remote blood pressure assessment comprising:
the camera recording at least 10 seconds of video at least 1000 frames per second;
segmenting the video into two non-overlapping segments;
averaging the reflectance signal for each channel in the video;
the step of processing the PPG signals comprises: applying smoothing filters; applying moving average filters, detrending the data, and cross-correlating the PPG measurements to find the time delay between each segment, and using the time delay to calculate the mean arterial pressure.

16. The system of claim 1, where an optical clearing agent is applied to the skin before recording of the images.

17. The system of claim 1, further comprising a means to measure distance from the target area to the camera, recording is automatically initiated once the target area is a pre-determined distance from the camera.

18. The system of claim 17, where the means to measure distance from the target area to the camera is a reference object.

19. The system of claim 17, where the pre-determined distance is selected to increase the robustness of the measurement of the useful information.

20. The system of claim 1, where image registration is used to remove motion artifacts received from the camera.
